(19) United States
(12) Patent Application Publication
(10) Pub. No.: US 2026/0232263 A1
(43) Pub. Date: Aug. 13, 2026

(54) WEARABLE DEVICE FOR PHYSIOLOGICAL MONITORING

(71) Applicant: INNOVATIVE HEALTH CONNECT PRIVATE LIMITED, Chennai (IN)

(72) Inventors: Arvind RAVICHANDRAN, Chennai (IN); Madhumita BALAKRISHNAN, Chennai (IN)

(21) Appl. No.: 19/150,565

(22) PCT Filed: Jan. 24, 2024
(86) PCT No.: PCT/IN2024/050066

§ 371 Date: Jul. 24, 2025

(30) Foreign Application Priority Data
Jan. 25, 2023 (IN) 202341005142

(51) Int. Cl. A61B 5/00; A61B 5/0205; A61B 5/024; A61B 5/318; A61B 7/04

(52) U.S. Cl. CPC A61B 5/6805; A61B 5/0002; A61B 5/02055; A61B 5/318; A61B 7/04; A61B 5/02427; A61B 2562/0271; A61B 2562/066

(57) ABSTRACT

The present invention relates to a modular wearable device for monitoring a user's health parameters. The present invention provides a better aid in remote monitoring, thus, improving the quality of life, aiding in recovery and improving the overall quantifiability of a person's health over intervals longer than prior possible. The present invention discloses a garment worn over the torso, within which multiple sensors are integrated in selective locations in order to best suit the needs of sensing/monitoring the functioning of the internal organs using non-invasive approaches. The present invention provides long-term monitoring of a patient's health parameters. The present invention provides a “smart-wearable” device with sensors, data processors, communication devices and their associated communication and energy circuitry.

### FIELD OF INVENTION

The present disclosure generally relates to physiological monitoring equipment and devices, and more particularly relates to a wearable device for acquisition and monitoring of vital parameters of a user.

### BACKGROUND OF THE INVENTION

In the last two decades, medical industry or wellness and fitness industry have seen drastic increase in patients or people who are in need of continuous monitoring of their vital parameters or other health parameters. For example, people with chronic or long-term illness, people under palliative care and pregnancy care, people with physical disabilities, people under post-operative care, geriatric patients or people undergoing rehabilitative recovery need their vitals to be monitored around the clock.

These requirements sometimes land up in hospitalization, even though hospitalisation may not be required that results in unnecessary occupation of hospital beds, thereby reducing the number of available beds for critically ill patients. Further, some patients who are being monitored under hospital environment may not need immediate medical intervention, thus leading to an imbalance between critical and non-critical requirements of hospitalization.

Further, during pandemics, the availability of hospital beds is also a critical point to be addressed because of the shortage of hospital facilities. In such situation, if the patient or person needs ‘round-the-clock’ monitoring, he/she has to be shifted out of hospital facilities and/or assisted living facilitics.

To cope with this issue and provide ‘round-the-clock’ monitoring to the patients or people with non-critical requirement of hospitalization, a possibility has been introduced in the form of a wearable device that eliminates need of connecting the patient or people to the bulky monitoring devices. Further, the above mentioned wearable device facilitates the monitoring of health parameters, regardless of location, thus reducing/eliminating the need for hospitalisation and its associated expenses Moreover, the patients in need of post-operative care may be better assisted with continuous remote monitoring using the wearable device

A conventionally known art discloses a physiological monitoring garment having a band-like configuration and incorporating respiratory, cardiac and physiological sensors. The garment is designed so that it is easily constructed from a few numbers of separate elements, and so that one garment design can be adjusted to subjects of a range of sizes and shapes. The design is further adapted to require little or no wearer effort during donning or wearer attention during use.

However, the limitation with such prior-art wearable devices is that they are merely able to report vital parameters that they monitor, to a wearer or user. For instance, most of the wearable devices monitor, in real-time, and report health parameters such as stress level and peak oxygen consumption.

The capability to simultaneously monitor multiple health and activity parameters continuously or across long periods of time without hindering the regular functions of the user or patient is absent with the existing wearable devices, thus, making them incapable of identifying illnesses or events in advance. Because of their inability of continuous monitoring of health or vital parameters, event precursors and early signs/indications often goes un-noticed and mapping of signs/indications get delayed and hindered. Also, conventionally known wearable devices lack the clinical accuracy and autonomy, so much so that, such devices require human intervention to raise an alert or initiate responsive action in critical cases. Thus, the absence of timely warning mechanism within the existing or prior art wearable devices results in delay of timely and potential life-saving interventions, especially in cases where a person requires a human intervention for administration of first-aid and the available medical support centre is far away.

There is therefore, a need in the art to provide a system and a wearable device capable of continuous monitoring of vital parameters including exertion level, peak oxygen consumption and other important vitals for longer period of time, analysis of said vital parameters in real time to detect signs of health issues, determine the severity of detected health issue and timely alert for the intervention of the emergency service providers.

There is also a further need to provide timely intervention and aversion of any critical situation of a person thereby minimizing the number of hospitalization requirement and reducing the overall cost incurred by a person due to healthcare.

### SUMMARY OF THE INVENTION

This section provides a general summary of the disclosure, and is not a comprehensive disclosure of its full scope of all its features.

An objective of the present invention is to provide a body wearable modular device that is capable of continuous monitoring of vital parameters of the user/wearer for longer period of time thus capable of being used for monitoring the user's health for abnormalities or assist the user in reaching fitness and athletic goals. Another objective of the present invention is to provide a solution capable of being used in multiple industries, including but not limited to medical, healthcare, athletic training or wellness industries to treat the user or patient from ailment.

Yet another objective of present disclosure is to provide a modular wearable device capable to monitor vital parameters including stress level, peak oxygen consumption and other important vitals for longer periods with time stamping and analysis of said vital parameters in real time to detect signs or indications of event or health issue and transmit said analysed data to a central server for further analysis and alert to the required destinations for early medical intervention.

A further objective of the present disclosure is to provide a modular wearable device that is capable of being integrated within a garment or a strap to be worn by the user to eliminate stress of wearing bulky wearable devices.

In consideration of the above-mentioned unmet need, the present invention provides for a device that is capable of being incorporated into a garment or a strap harness form-factor to provide ‘round-the-clock’ vital monitoring without sacrificing user mobility. The garment or the strap harness form factor of the present invention enables everyday usability with minimal effort.

In an embodiment, the device for measuring physiological parameters of a user, the device comprising a sensor unit having a plurality of sensors in one or more sensor types to sense one or more physiological parameters of a user, a control unit coupled with the sensor unit, the control unit configured to receive one or more sensed signals from each of the plurality of sensors, a communication module coupled with the control unit, the communication module configured to establish communication between the control unit and a computational server, an amplifier unit coupled with the control unit, the amplifier unit configured to amplify audio signals received from at least one audio capturing instrument, a battery receiver and power regulation module coupled with the control unit, the sensor unit and the battery unit, wherein the battery receiver and power regulation module controls powering, charging and protection of components of the device, wherein the control unit is configured to receive physiological signals of the user through the sensor unit and process the same to provide values against each of the physiological parameters to the remote monitoring unit in real-time, wherein the device is adapted to be integrated in a wearable garment with all components of the device embedded within said wearable garment or strap harness/strap vest to measure at least one of heart rhythm and electrical activity, triaxial linear and angular motion, core body temperature and blood pressure.

In an embodiment, the sensor unit comprises PPG sensors, ECG sensors, CBT sensors and IMU sensors.

In an embodiment, the control unit comprises at least one dedicated sub-control unit for at least one of the sensor types.

In an embodiment, the control unit comprises at least one filter controller and one clock generator.

In an embodiment, the amplifier unit comprises at least one analog to digital converter, wherein the amplifier unit is configured to receive audio signals from at least one of a full pick-up piezo-microphone or a full pick-up electret-microphone and at least one of a piezo-mic stethoscope and an electret-mic stethoscope.

In an embodiment, at least one of the piezo-mic stethoscope and electro-mic stethoscope is embedded into the wearable garment or strap harness/strap vest.

In an embodiment, the control unit includes at least one alert generation module configured to raise an alarm in case at least one vital parameter reading is beyond an acceptable range for a predefined time period.

In an embodiment, the modular wearable device may include provision of a sensory feedback to the user/wearer in the form of optical emission feedback (LEDs) or haptic technology for an improved user convenience.

In a further embodiment, the modular wearable device may include a user-controlled mechanism which enables the user to define certain functionalities of the device and control the alert generation capabilities.

Also described herein is a clothing having the device as summarized above, the device embedded within the clothing, the clothing adapted to cover a torso of a user and is configured to accommodate components of the device configured to acquire, monitor and transmit one or more physiological and motion related information of the user to a computational storage server.

In an embodiment, the sensors are positioned strategically in the clothing and are sealed in a waterproof manner.

In an embodiment, the sensors are positioned in manner to achieve Triangulation capabilities and an internal reference plane virtualisation system.

The present invention achieves its objective of a modular wearable device configured to provide a non-invasive observatory care capable to be deployed at any geographic location of the user or patient. The present invention does not restrict the mobility and also ensures comfort of the wearer. The proposed solution by the present invention is capable of monitoring multiple parameters without the need of specialized hardware and also ensures higher level of accuracy, fidelity of one or more vital information thereby allowing a user to seek necessary health intervention. Thus, the proposed solution effectively reduces the logistical burden on both entities: the user and the healthcare system.

The proposed solution can also provide a long-term analysis of the health parameters using pattern and trend analysis, instantaneous response and alert generation to emergency situations such as cardiac arrest or other life-threatening events. Further, the proposed solution may also be made to communicate with other compute devices to store and process data for both short- and long-term usage. Even further, the proposed solution is capable to periodically take vital reading without human assistance thereby providing a constant medical care while not hampering everyday activities. The present invention also provides the ability to be powered and functioned remotely allowing a cross-validation of the alerts generated.

Thus, the present invention is able to minimize the time taken for the emergency signal to reach the respective personnel. The invention is also able to provide the ability to record and analyse long-term data ‘round-the-clock’, a feature which was unavailable previously. The proposed solution also requires a basic or minimal training and is also configured to be remotely accessed for troubleshooting as opposed to conventional equipment. The proposed solution by the present invention is further capable to monitor variations in health parameters theroby supporting an improved diagnosis as well as recovery of the user/patient.

Additional aspects and advantages of the present disclosure will become apparent to those skilled in this art from the following detailed description, wherein only illustrative embodiments of the present disclosure are shown and described. The present disclosure is capable of other and different embodiments, and its several details are capable of modifications in various obvious respects, all without departing from the disclosure. Accordingly, the drawings, description and examples in this summary are intended for purposes of illustration only and are not intended to limit the scope of the present disclosure.

### BRIEF DESCRIPTION OF DRAWINGS

A better understanding of the feature and advantages of the present invention will be obtained by reference to the following detailed description that sets forth illustrative embodiments, in which the principles of the invention are utilized, and the accompanying drawings or figures, of which:

FIG. 1 shows a block diagram of the modular wearing device in accordance with an embodiment of the present disclosure.

FIGS. 2A-2C illustrates the modular wearing device in a male-form of the garment form-factor, in accordance to an embodiment of the present invention.

FIGS. 3A-3C illustrates the modular wearing device in a female-form of the garment form-factor, in accordance to an embodiment of the present invention.

FIGS. 4-8 illustrate construction of the sensors included in the modular wearing device in accordance to an embodiment of the present invention.

### DETAILED DESCRIPTION

The embodiments herein and the various features and advantageous details thereof are explained more fully with reference to the non-limiting embodiments that are illustrated in the accompanying drawings and the following description. Numerous variations, changes and substitution may occur to those skilled in the art without departing from the invention. It should be understood that various alternatives to the embodiments of the present disclosure herein may be employed.

For the case of reference, certain terms used in this application and their meanings as used in this context are set forth. To the extent a term used herein if not defined below, it should be given the broadest definition persons in the pertinent art have given that term as reflected in at least one printed publication or issued patent. Further, the present techniques are not limited by the usage of the terms shown below, as all equivalents, synonyms, new developments, and terms or techniques that serve the same or a similar purpose are considered to be within the scope of the present claims.

The articles “a” and “an” as used herein mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

The term “Clinical accuracy” used herein means clinically accepted accuracy and is a subjective standard that is susceptible to variations with changes in demography, geography and environmental factors including but not limited to temperature and pressure.

The term “Long-term” medical and/or recovery intervention used herein refers to any duration of hospitalisation that lasts for a longer period of time.

The present invention as disclosed herein below is directed towards a wearable physiological monitoring device capable of continuous and autonomous monitoring of vital parameters of a user such as heart-rate, oxygen saturation, cardiac activity, body temperature, blood pressure, physical activity, step count tracking, breathing rate, peak oxygen consumption and other important vitals as well as general wellness for longer period of time, analysing said vital parameters in real time to detect signs of any health issues, and determining the severity of a detected health issue along with the capabilities to timely alert emergency service providers for any medical intervention that may be required.

There is also a further need to provide timely intervention and aversion of any critical situation of a person thereby minimizing the number of hospitalization requirement and reducing the overall cost incurred by a person due to healthcare.

The present invention therefore provides a non-invasive observatory care with long-term medical or recovery care. The present invention also provides for a portable and domestic use monitoring equipment with clinical accuracy and autonomy with minimum human intervention in the form of a wearable clothing that provides for closer, deeper and more precise monitoring of the vitals and/or wellness of a user compared to other wearable devices available in the market, for example in the form of wrist bands.

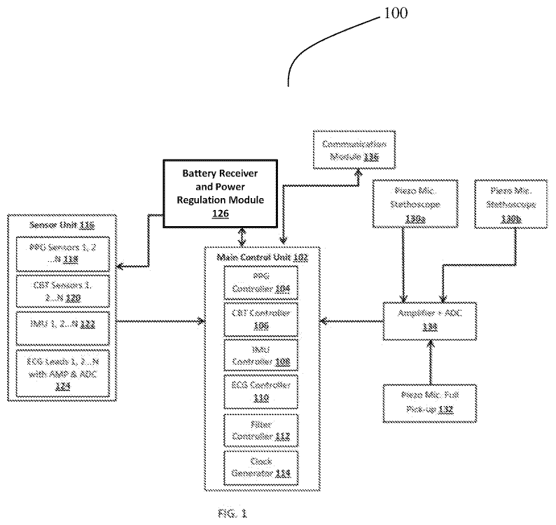

FIG. 1 illustrates a block diagram of a modular wearable device 100, in accordance with an embodiment of the present disclosure, wherein a controller component of the present invention, interchangeably referred to as a control unit or main controller 102, enables and manages the device 100 macro functionalities. The main control unit 102 comprises one or more controller units 104, 106, 108 and 110, which either collectively or singularly control operations the modular wearable device 100. The device 100 also includes a sensor unit 116 having a plurality of sensors 118, 120, 122, 124 which are communicably connected to the one or more controller units 104, 106, 108, 110 of the main controller unit 102. The main control unit 102 thus, controls the operation, internal flow of signals, data and commands, of some or all of the sensors integrated in the device. The sensor unit 116 having one or more of the sensors 118, 120, 122, 124, are configured to assess the state of health of the wearer by detecting (sensing) different vital parameters of a user. The various sensors that are part of the sensor unit 116 may include a plurality of Photoplethysmogram (PPG) sensors 118, a Core Body temperature sensor 120, an inertial measurement sensor unit (IMU) 122 and electrocardiogram (ECG) Electrodes 124.

In an another embodiment, the device 100 further includes a battery receiver and power regulation module 126, connected to the main control unit 102, and the sensor unit 116, and is configured to manage power consumption and battery usage of the device 100.

In a further embodiment, the device 100 also includes a full pick-up piezo-microphone (mic) or a full pick-up electro-microphone 132, and one or more piezo-microphone stethoscope or an electret-mic stethoscope 130a, 130b, which are communicably connected to the main control unit 102.

In a further embodiment, the device 100 includes a communication module 136 communicably connected to the main control unit 102, and is configure to communicate or send signals to a server (not shown here) and/or a user device (not shown here).

The invention comprises of multiple sensors that detect the same vital parameter from different perspective, which provides Internal cross validation of values and ensure data quality and reliability. A detailed description of the various sensors included in the disclosed device 100 is provided in following paragraphs.

In an embodiment, one or more PPG sensors 118 are included in sensor unit 116 of the present invention which may individually communicate with the main controller 102 through a bi-directional data line. The PPG sensor 118 is integrated at its most ideal location, which is at the wrist of the wearer or further towards the extremities from the wrist of the wearer, where the epidermal thickness is less. The present invention includes two Photo-plethysmography sensors 118 (one for each wrist) and its auxiliary electronic components, which are housed in an enclosure and placed on the garment's/strap's wrist area. The enclosure contains a transparent membrane that allows light to pass from the sensor to the human skin and vice-versa. This transparent membrane also contains a flexible gasket around it to isolate the light signal from the external light. Moreover, the enclosure is attached firmly under pressure with the human skin to ensure signal conformity under all circumstances.

The Photo-plethysmography sensor 118 is configured to detect heart-rate of the end user by measuring fluctuation in blood pressure over time of the wearer. In addition to the above, the Photo-plethysmography sensor 118 is also configured to detect Blood Oxygen percentage by measuring the oxygen cell count in blood using light technique thereby providing an accurate count of the Blood Oxygen percentage.

The device 100 also includes a Core Body Temperature sensor (CBT) 120 or simply the temperature sensor 120, configured to monitor a body temperature of the user/wearer. Persistent and continuous monitoring of the temperature trends may allow early detection of multiple ailments, chronic illnesses and conditions like sepsis, which may not be immediately and overtly observable. The CBT sensor 120 is configured to detect Hypothermia by monitoring the body temperature and comparing it with external temperature.

The temperature sensor 120 construction requires its active face to be in direct contact with the target surface directly or through the use of a thermally conductive interface that transfers energy to it for which temperature is to be measured. Keeping in mind the wearer's comfort, the CBT sensor 120 has been integrated with one of the ECG leads and in conjunction with a thermally conductive strip intended to carry the body heat from the armpit area to the location of the sensor to perform the same function. The location of the CBT sensor 120, thus, captures and accurately displays core body temperature of the user.

In a further embodiment, the device 100 includes a motion tracker sensor or IMU sensor 122 which is configured to constantly track motion and activities of the user. The IMU sensor 122 are calibrated to detect sudden movements and to clearly differentiate fall and impact. Specifically, the IMU 122 includes a ‘Micro Electro-Mechanical Sensor’ unit, which tracks motion with respect to and as a component function of the three Cartesian co-ordinate axes. More specifically, the IMU 122 measures the motion of the wearer as a function of time and acceleration (both positive and negative) split across its constituent values resolved as acceleration values along the three primary Cartesian axes.

In an alternate embodiment, the sensor unit 116 integrates a cluster of three IMUs to provide for Triangulation capabilities. This helps in capturing information necessary for identifying and analysing information pertaining to posture, gait, sedentary orientations and other parameters concerning relative position of torso. The IMU sensor(s) 122 are placed in strategic places such that it can accurately detect the user's position and orientation and adjust readings accordingly. The device 100 is provided with an internal reference plane virtualisation system using the orientation of gravity as the absolute standard. The IMU 122 sensor's position enables the device to create a reference plane which is cross-linked with wearer's body orientation for error correction. Further, the individual IMUs are integrated alongside three of the four ECG pick-up points, kept as apart as they can be, to ensure least reference plane distortion, thereby solving the relative position issue. The IMU sensor 122 is turned on continuously in order to record readings and transmitting continuously to enable live activity monitoring.

In another embodiment, the disclosed device 100 includes an ECG unit 124 having one or more ECG electrode(s) along with their auxiliary electronics, included into a single unit, which are encased in a watertight, permanently sealed enclosure attached to the garment/or strap vest. The electrode(s) of the ECG unit is exposed on the skin facing side to ensure electrode-skin contact. Further, the ECG 124 enclosure has a hole passing through each of the four units to act as a passage for injection of an electrolytic gel to reduce electrical impedance at the point of contact. The ECG 124 electrodes are configured with concentric grooves to ensure retention of said electrolyte gel and ensure proper contact even in the event of relative motion between the electrode and the wearer's skin. The present invention also provides an on-board clock and its alarm system sends an alert signal to the ECG unit 124 at pre-determined time interval and it triggers the ECG unit 124 to take readings accordingly. The ECG unit 124 is configured to detect complete heart rhythm in order to cross validate the user's heart rate. The ECG unit 124 has the capability to periodically take user's Electro-Cardiography output at pre-determined regular intervals.

The ECG 124 electrodes may be provided with an amplifier and an analog-to-digital convertor (ADC), in order to counter any electrical interference faced by the ECG 124 leads from nearby components and/or devices. Each voltage line corresponding to each of pick-up point is, therefore, connected to a dedicated amplification module to prevent signal cross talk and perform differential amplification. The ECG hardware in its entirety has been electrically shielded and isolated from the outside as well as from other parts of the same system and has been designed in such a manner that the downscaling of the ECG system to a wearable form factor or its integration in a garment or strap harness/strap vest does not affect the reliability of the output.

The present invention also provides an on-board clock and its alarm system sends an alert signal to the ECG unit 124 at pre-determined time interval and it triggers the ECG to take readings accordingly.

In an embodiment, the device 100 is designed to prevent interference to the highly sensitive ECG pickup points. In order to keep operations of the ECG 124 and PPG 118 components exclusive with respect to one another, the power and data lines of the PPG sensor 118 are given distinct pass-through within the ECG pickup points.

In another embodiment, the device 100 also includes a full pick-up piezo-microphone or a full pick-up electret-microphone 132. The microphone 132 and amplifier circuits are placed close to the organ(s) of interest and frequency-tuned to detect and transmit the sounds of internal organs, especially cardiac and pulmonary sounds. The microphone 132 is configured to capture fall sounds and is used to cross validate the fall data from the IMU 122 output. Further, the sensitivity of the microphone unit 132 enables it to pick up impact forces and is calibrated to differentiate it from internal organs' sound.

In yet another embodiment, the device 100 is also configured to receive inputs from one or more Piezo microphone stethoscope or an electret-microphone stethoscope 130a, to capture various bodily sounds. These sounds as captured by these piezo/clectro components may be amplified and converted by an amplifier-cum-ADC circuit 134 and the output may then be provided to the control unit 102.

All of the sensors described above collectively comprise the sensor unit 116 which is further communicably connected to the control unit 102. The following paragraphs describe the control unit 102 in detail.

The control unit 102, as provided in the present invention, is configured to manage flow of data from individual sensors by either saving it locally for a limited amount of time or transmitting it live to a cloud server. The control unit 102 includes at least one of a PPG controller unit 104, a CBT controller unit 106, an IMU controller unit 108, ECG controller unit 110, and Clock generator unit 114. The control unit 102 is further configured to manage timed activation and deactivation of individual components according to extrinsic commands. The control unit 102 receives, processes, stores and executes external commands or instruction-sets.

As illustrated in FIG. 1, the modular wearable device 100 includes a battery receiver and power regulation module 126 which is combination of a battery unit and a Battery Management System (BMS), configured to maintain a constant power supply to the entire device 100, for a specified period of time without relying on external means.

In an embodiment, the battery unit of the battery receiver and power regulation module 126 of the present invention preferably uses lithium chemistry batteries in a pouch cell(s) form-factor, and preferably uses two of the same in order arranged in parallel manner to obtain desired power output and higher charge (life) expectancy. The battery pack also embeds ejector pin setup and a quick release mechanism used to eject the battery pack form the invention. In order to protect the battery unit 126 from being shorted as well as contain the necessary safety standards such as over/under current/voltage/temperature protection while charging and during usage, a software driven handshaking procedure has been integrated with the device to ensure that the battery's function is not initiated unless the battery is safe to use and the target device has been identified to be of the appropriate specification.

In an embodiment, the BMS of the battery receiver and power regulation module 126 may include a Monolithic-PCB integrated inside the battery receiver and power regulation module 126 and permanently mated with the battery cells. The BMS controls charging, discharging and status reporting of the battery pack and ensures that the cells have a layer of functional protection. The BMS is also configured with numerous safety systems in it, such as over-voltage protection, under-voltage protection, over-current protection and over-temperature protection, which ensure that the battery doesn't experience failure due to thermal runaway if the terminals are shorted. Additionally, the BMS is configured to prevent the battery from catching fire even in the event of a deliberate short circuit. The BMS is also configured to include any necessary software update for the entire system without the requirement for changing any hardware component.

The battery receiver and power regulation module 126 that is designed to house the batteries and the BMS also includes vent holes to avoid the battery from overheating.

Further, the battery receiver and power regulation module 126 may include a distribution plane, responsible for transferring the power from the battery receiver and power regulation module 126 and distributing it to individual components based on demand, and a Buck converter (not shown), designed specifically to step down the voltage received from the battery unit to levels optimum for individual component or components operational in the device 100.

In operation, power is supplied from the battery unit via the BMS circuit of the battery receiver and power regulation module 126 and is further forwarded to the various components by the distribution plane. In an embodiment, components that operate on same voltages are grouped together in a single power rail which gets its power from the distribution plane via a dedicated Buck converter. The buck converter steps the voltage down to desired operational level and before sending it through the corresponding power rail.

Further, the invention is provided with the power and communication protocols independent for each functional sub-system, thereby allowing the individual sensors or functional modules to be completely powered off or remain persistently powered on. Also, the electronic components that make up the invention are selected in such a manner that either their power consumption is low or they have a separate power saver mode, which aids in improving the invention's power efficiency. Thus, the design of the battery receiver and power regulation module provides certain components to be turned off and on based on their operating time

As illustrated in FIG. 1, the present invention also includes a communication module 136 for providing connectivity to a server or other computing devices outside the device 100. The communication module 136 enables generation of an alert to healthcare professionals when the device 100 detects a critical situation. In order to ensure connectivity in every situation, the invention provides one of two different forms of connectivity forms of connectivity, Wi-Fi connectivity or Long-Term Evolution or LTE connectivity, thereby providing connectivity under different operating condition(s).

The device 100 provides a low-profile connector to carry the communication signal from the device's main controller 102, to the Wi-Fi module 136. The dedicated Wi-Fi module 136 configured to provide connectivity especially when the invention is in a confined environment. Such connectivity is useful when multiple garments or strap barnesses/strap vests are in the same confined operating space, such as bulk purchases are made, and/or operate under the same network, such as care homes and hospitals.

The construction of the present invention ensures the Wi-Fi module 136 and the antennas are kept safely away from other components of the device to avoid signal hysteresis. Further, the Wi-Fi module 136, and its antenna are shielded from other devices to preserve the data transmitted through the antenna. Also, the antenna used in the invention's communication module 136 is well-protected against physical damage to ensure constant connectivity, and is water-proof to protect its electrical and communication integrity.

In another embodiment, the invention further provides connectivity in the form of Long Term Evolution (LTE), to ensure connectivity when the wearer is outside of a confined environment, and is preferably attached to the battery receiver and power regulation module 126 which comes out along with the battery receiver and power regulation module 126 when removed. The battery receiver and power regulation module 126 of the invention comes with an inbuilt LTE capable sim card as standard, and the battery receiver will contain the LTE modem module to ensure shortest possible communication distance. The LTE connectivity is configured to ensure that SIM is close to modem for enhanced signal strength, and is resistant to water and other contaminants from the external environment to function properly.

In an alternate embodiment, the antenna provided in the present invention receives any OTA updates and sends the signal to the microprocessor which runs the update autonomously without the need for a technical person.

The following paragraphs describe the garment construction and placement of components, namely layout, component placement, substrate, stiffeners, structural supports.

The invention has the capability to be used in two different form factors, namely in the garment form-factor and in the strap/harness/strap vest form factor. In the garment form-factor it is preferably in the form of a full-sleeve T-shirt having separately a male garment form-factor and a female garment form-factor that houses all the sensors and sub-electronic systems in it, which are manufactured using an advanced manufacturing technique. In both of the form-factors all the sensors and sub-electronics are placed with the help of retainer straps.

One of the objectives of the invention is to make the garment washable to make the garment everyday usable. Hence, protecting the sensors and their associated electronic components plays a major role in making the garment everyday usable. This is achieved by permanently encasing the sensors and their associated electronic components in a water-tight enclosure. The various auxiliary modules such as, Main controller 102; Battery receiver and power regulation module 126; Connectivity module 136 are constructed to ensure waterproofing.

In order to affix all the electrical contacts to the body, the present invention bas employed a dedicated fabric design that ensures the ECG electrodes to return to their desired pickup points during ideal condition and in operation. The device 100 is incorporated in a conductive material, for example the PCB, and the entire region carrying the parts responsible for ECG signal processing are encapsulated in an EMI shielding ‘Via (1-noun)’ array and is grounded. The various conductors and electrical leads are configured to conform to the user's body contours.

Further, to make the wearable device power efficient, the present invention includes a system for optimising the power consumption of all the internal components by integrating the use of ‘timed operation’ and ‘intermittent operation’ functionality. The invention is configured to function on a schedule, which is pre-configured and also provides capability to be re-configured based on the wearers' requirement. The invention also provides provision to completely shut power off to parts of the system which are not in use. The electronics of the individual sensor and sub-system is configured to be shut down remotely via a secured pathway. The invention provides has a built-in internal memory buffer unit that is configured to store data during loss of connectivity and send it back when connectivity is restored.

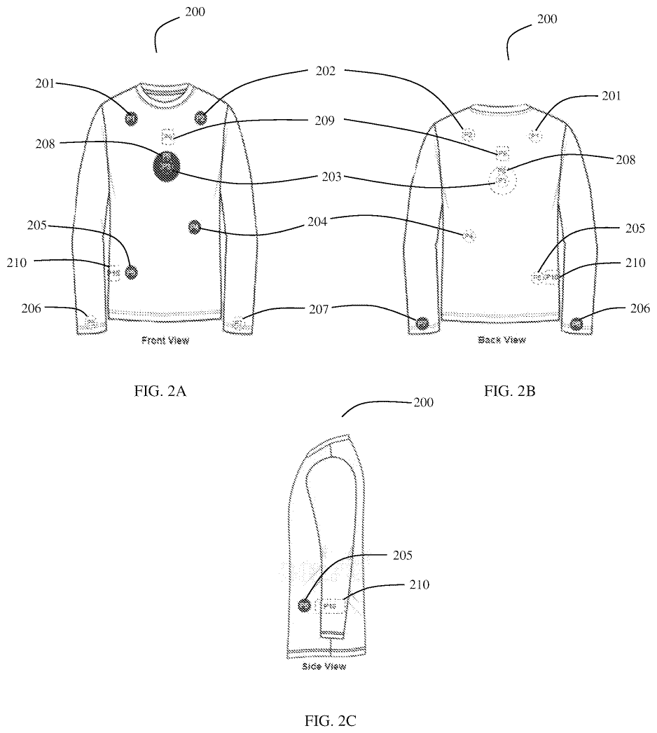

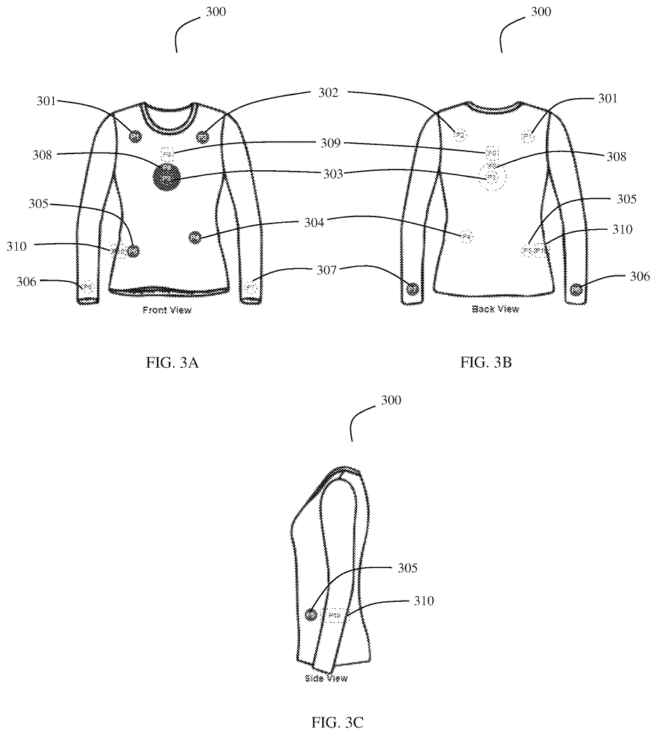

FIGS. 2A-2C illustrate a male form 200 of the garment form-factor designed for male users or patients, in accordance to one of the embodiments. While, FIGS. 3A-3C illustrates a female form 300, of the garment form-factor, in accordance to an embodiment of the present invention. The garment 7 or more enclosures or designated points, shown as 201 to 210 in FIG.(s) 2A-2C, and shown as 301-310 in FIG.(s) 3A-3C, that are permanently attached to it.

In FIGS. 2A-2B and 3A-3B shows point 1 (P1), 201 and 301, consisting of a Temperature Sensor 120, a 3-D motion sensor (IMU), 122, and a plurality of ECG electrodes, 124, all placed below or within rib cage frame. The temperature sensor 120 in the assembly is located in a position where it makes direct body contact. The 3-D motion sensor (IMU), 122, in the assembly is one of the three motion sensors provided in the present invention to detect the body motion and angle, thus, being able to track wearers' movement and detect fall. The ECG electrode 124, present in the assembly is one of the 4 electrodes provided in the invention and is capable of acquiring a reading of the wearer which is equivalent to 6 ECG leads reading.

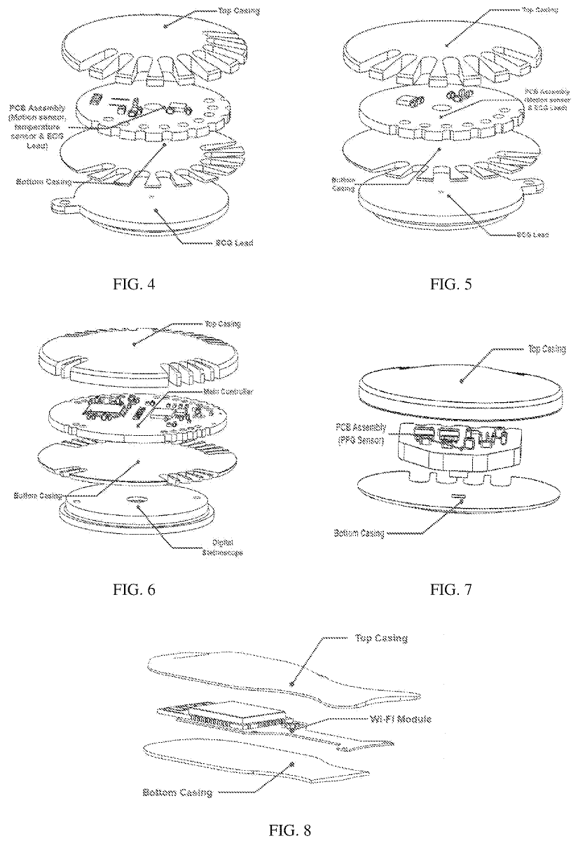

FIG. 4 illustrates an arrangement of sensors to be attached to the point P1 201 in accordance to an embodiment of the present invention. In order for the position P1, 201 and 301, to include all of three sensors, 120, 122, and 124, the sensors 120, 122, 124 are placed on a single hard PCB board which is further encapsulate in a silicone capsule and placed in the position P1, 201 and 301. Such arrangement allows the present invention to obtain the desired vital parameter output at the desired level of accuracy. The construction of the above said assembly is depicted in the below image.

Point 2 (P2), 202 and 302, as illustrated in FIGS. 2A-2B and 3A-3B, shows its placement with respect to the male and female garment form-factor. Point P2, 202 or 302, is located under left clavicle near left shoulder within the rib cage frame, whereas Point 4 (P4), 204 shown in FIGS. 2A-2B and 304 in FIG. 3A-3B, is located below pectoral muscles lower edge of left rib cage. Both of the points P2 and P4, 202 or 302, and 204 or 304 respectively, includes one of the three 3-D Motion Sensor (IMU) 122, provided in the present invention, and 4 ECG electrodes, 124. The 3-D Motion Sensor (IMU), 122, used at P2, 202 or 302, and P4, 204 or 304, is configured to work homogenously with other motion sensors in order to accurately record the wearer's activity and detect fall. On the other hand, the ECG electrode, 124, included in positions P2, 202 or 302, and P4, 204 or 304, is one of the four electrodes provided in the present invention configured to record 6 lead ECG reading of the wearer.

The device construction includes an identical construction, illustrated in FIG. 5, to be included in both of the points P2, 202 or 302, and P4, 204 or 304. The motion sensor, 122, and ECG electrode, 124, are placed on a single hard PCB board that is encapsuled in a silicone enclosure.

Both point 3 (P3) 203 and point 8 (P8) 208, illustrated in FIG.(s) 2A and 2B, and 303 and 308 illustrated in FIG.(s) 3A and 3B, are located at the centre of the chest over the sternum. The point P3 203 or 303, includes at least one digital stethoscope 130a or 130b placed in a direct skin contact with the user/wearer, thereby, ensuring cardio/pulmonary sounds being captured at desired quality level. Point P8 208 or 308, includes the main controller 102, and the Wi-Fi module 136, as an auxiliary unit to the main controller unit 102, in accordance to one of the embodiments of the disclosed invention. The main controller module 102, is placed directly on the digital stethoscope 130a or 130b, and mechanically linked together.

As illustrated in FIGS. 2A-2B and 3A-3B, points P3 203 or 303, and P8 208 or 308, are sand-witched together in accordance to one of the embodiments of the disclosed invention. FIG. 6 illustrates the construction of the above said assembly in accordance to an embodiment of the present invention.

Point 6 (P6) 206, as illustrated in FIG.(s) 2A and 2B, and 306 as illustrated in FIG.(s) 3A and 3B, is located at the inner side of the right wrist, whereas point 7 (P7) 207, located at the inner side of the left wrist. Points P6 206 or 306, and P7 207 or 307, includes a PPG sensor 118, positioned such that the light emitting and receiving elements are facing the skin and also in direct physical contact. The present invention provides two PPG sensors 118, in total and working in tandem to acquiring the Blood oxygen saturation as well as heart rate of the wearer.

The construction, as illustrated in FIG. 7, for sensors provided in points P6 206 or 306, and P7 207 or 307, is identical. The PPG sensor 118 is placed on a single hard PCB board and positioned with its lighting sourcing and receiving element facing the skin. The entire assembly (PCB and PPG sensor 118) are encapsuled in a silicone enclosure. The silicone enclosure has an opening for the light from the PPG sensor 118, to directly fall on the skin. The entire assembly is pressed against the skin with the help of a constant pressure using a retainer. This holds the sensor firmly to the skin ensuring signal accuracy and quality while also shielding the PPG light signal from external interference.

Point 9 (P9) 209, as illustrated in FIGS. 2A-2B, and 309, as illustrated in FIG. 3A-3B is placed at the centre of the clavicle bones above P8 208 or 308, which includes a Wi-Fi (communication module) 136, and its antenna. This antenna transmits vital signals to a central server where all the collected data is further processed to ascertain the wearer's state of health. The communication module 136 assembly, as illustrated in FIG. 8, is enclosed inside a silicone capsule that protects against water damage and damage from other external mediums. Further, FIG. 2A-FIG. 2B and FIG. 3A-3B, illustrates point P10 (P10) 210, 310, is placed on the lateral side of the lower right abdomen, which includes a pair of batteries and a housing for integration of the batteries to the garment or strap harness/strap vest. The battery receiver position aligns with the curvature of the body thus causing least discomfort to the user/wearer.

TABLE 1

| Point | Electronics | Position |
|---|---|---|
| P1 | Temperature Sensor, 3-D | Under right clavicle near right shoulder |
| | Motion Sensor, 6-Lead ECG | within rib cage frame |
| | electrode | |
| P2 | 3-D Motion Sensor, 6-Lead | Under left clavicle near left shoulder |
| | ECG electrode | within the rib cage frame |
| P3 | Digital Stethoscope | Centre of the chest over the sternum |
| P4 | 3-D Motion Sensor, 6-Lead | below pectoral muscles lower edge of |
| | ECG electrode | left rib cage |
| P5 | 6-Lead ECG electrode | Lower right abdomen |
| | (Reference) | |
| P6 | PPG Sensor | Inner side of the right wrist |
| P7 | PPG Sensor | Inner side of the left wrist |
| P8 | Main Controller | Centre of the chest over the sternum |
| P9 | Wi-Fi Module | Centre of the clavicle bones above P8 |
| P10 | Battery receiver and power | Lateral side of Lower right abdomen |
| | regulation module, LTE Module | |

Table 1 summarizes all the points (P1-P10), the sensors included at those point and their corresponding placement provided in the garment form-factor of the present invention.

The present invention is configured to accommodate most of the components of the garment within an enclosure. However, some components are placed outside to achieve their desired functionality. For instance, the Wi-Fi module's antenna is placed above the sixth enclosure (i.e., above the Main controller module (102)). The ECG reference electrode is placed above the right hip bone relative to battery pack's position.

To prevent any data loss due to material resistance on the garment and interference, the present invention is configured to shift the data line to a higher voltage before transmission, which is then stepped down at reception.

In an embodiment, a top cap and a bottom body half of the battery receiver and power regulation module 126 are permanently sealed using a water proof adhesive. The gap and volume that exist between the BMS board, its connector, and the battery pack assembly's top cap are also filled with a water-proofing material to act as an ingress barrier. Further, the space between the battery assembly and distribution plate is protected from water ingress by integrating a compression seal formed by the mating of the two parts (battery pack and receiver assembly).

In an embodiment, the device 100 is incorporated in a conductive material and fabric to ensure shielding from EMI and ensure isolation of signals outside the PCB. With regards to the PCB, the entire region carrying the parts responsible for ECG signal processing are encapsulated in a EMI shielding ‘Via (1-noun)’ array and is grounded.

Further, the electronics and PCBs of the system are integrated into the fabric to create a garment form-factor or a strap harness/strap vest form-factor, as may be more suitable in terms of a user's comfort. The various conductors and electrical leads are configured to conform to the user's body contours.

In order to make the wearable device power efficient, the present invention includes a system for optimising the power consumption of all the internal components by integrating the use of ‘timed operation’ and ‘intermittent operation’ functionality. The invention is configured to function on a schedule, which is pre-configured and also provides capability to be re-configured based on the wearers' requirement. The invention also provides provision to completely shut power off to parts of the system which are not in use.

With regards to the manufacturing of the provided garment form-factor, a knit garment is used as the base to which all the other components are installed. The garment has the property of being bio-compatible, anti-microbial and hypo-allergenic in nature. The garment is manufactured with a blend of stiff and ordinary fabric and conductive yarn in areas where electrical conductivity is needed.

The garment form-factor additionally includes stiffeners not only to ensure body-garment contact conformity but also to distribute the load evenly and thus ensuring wearer's comfort. On the whole stiffeners are placed in three places. They are placed on each wrist to ensure contact between the PPG sensor 118 and skin. Next stiffener is placed near the hip bone and it carries the battery assembly. Another stiffener is placed near the neck and clavicle bone. The invention in its garment form factor may also include additional stiffeners, wherever necessary to ensure it helps the garment to adhere to the wearer's torso firmly

The below paragraphs described the functional sub-routines of the present invention.

The present invention is configured such that the entire device can be remotely turned on and off in case of necessity. The invention is capable of both remotely controlled and autonomous operation and has the ability to receive and execute commands. The device is also configured to run self-diagnosis at regular interval as well as when requested. The device also includes the necessary communication protocols to ensure all internal systems and modules are capable of receiving and responding to commands. In addition, the device is configured to send error reports when requested by compiling all the currently available independent system errors to the appropriate repository.

The garments/devices are configured to communicate with one or more data servers, from where it can be viewed or transmitted. The invention is further configured to execute diagnostic commands on individual sensors or functional modules. Further, each sensor and functional module are configured and connected in a manner to allow independent functioning, communication and diagnosis.

The present invention is configured to eliminate external interferences and isolate the reading recorded to prevent data skewing by shielding electronic components that are sensitive to external interferences with a shielding material and by using special cables in certain areas to reduce the amount of signal interference.

The invention allows record of reading at a pre-set time. The present invention provides an internal clock which acts like an alarm. The electronics are configured to receive signals from the alarm and take readings accordingly. The below paragraphs discuss the various ranges with regard to the alert mechanisms.

In an embodiment, the invention is designed with an in-built alert and emergency trigger mechanism that helps the wearer get the medical professional's attention at the earliest possible time. Most of the vital parameter monitored by the invention has a pre-determined acceptable range, alert range and an emergency range that varies according to individual's several factors namely age, height, weight, pre-existing medical condition, lifestyle habits and so on. Generally, when a vital parameter value is within the acceptable limit, it is considered normal. When the parameter value goes to the alert range the alert mechanism is triggered from the hardware and for the period of time the parameter is in that range is recorded and the data is sent to the server.

When the parameter stays in the alert range for more than a pre-set time, or if the value moves to the emergency range, it is considered as an emergency and the emergency mechanism is triggered. The emergency mechanism generates a warning signal to the wearer's doctor, nearby EMT (Emergency Medical Technician) team and to the wearer's next of kin (person who takes care of the wearer).

With regards to the oxygen percentage present in the blood, the present invention provides for an alert mechanism that works only in one direction i.e., the alert and emergency mechanism are triggered only when the value falls below a pre-set range. Specifically, when a person's blood oxygen percentage is above the highest point of the alert range, it is considered normal. When the value, goes below and falls in the alert range, it indicates the wearer's health is in concern. In such an event the invention generates the alert mechanism and records the event. If the value persists to be in the same region for more than a pre-defined time or if the value reduces further below the alert range, it denotes the oxygen level of the wearer as low and generates the emergency alert to respective professionals.

For the CBT measure, the alert mechanism works in two directions, i.e., the alert and emergency mechanisms are triggered when the value is below or above a pre-set range. When the value is within the acceptable limit, then it is considered normal. When the value drops below the acceptable limit, it generates an alert mechanism and records the event. When the value stays in the same limit for more than a pre-set time period or if the value drops further below, the risk of the wearer getting hypothermia becomes significantly high, thereby, the emergency alert mechanism is generated and sent to the respective professionals.

The same holds true when the value increases. When the value is highest than the acceptable limit, the alert mechanism is raised and the event is recorded. If the value stays in the same range for more than the pre-defined time or if the value rises beyond the alert range, the possibility of the wearer getting a thermal shock is high and accordingly, the emergency alert mechanism is triggered and sent to the respective professionals.

The present invention is additionally configured for motion tracking of the wearer. The wearer's activity level is monitored to detect any impact and falls. The IMU 122 sensor's output detects sudden movements and impacts and combined it with the accelerometer's output, enabling to detect a fall. Based on the wearer's age and underlying health condition and surgical history the alert mechanism is set only for the vulnerable people. When a vulnerable person wears the invention and falls or has a severe impact, the invention's IMU 122 sensor picks it up and compares it with the accelerometer value. If the output complies with the pre-defined condition, it is considered as a fall/impact and emergency mechanism is generated to the respective professionals.

Heart Rate Monitoring is achieved using the Photo-plethysmography (PPG) sensor 118 used for Blood Oxygen percentage monitoring. Its alert mechanism is bi-directional, similar to Core Body Temperature. When the heart rate is within the acceptable limit, it is considered normal. However, when the heart rate is above the acceptable range, it sets the alert mechanism and records the event. When the value stays in the same range for more than the pre-defined time or if the value exceeds the alert range, the possibility of the wearer getting a heart attack significantly increases, thereby triggering the emergency mechanism and sending an alert to the respective professionals. Similarly, when the heart rate falls below the acceptable limit, the emergency mechanism is triggered and the event is recorded. When the value is in the same range beyond the set time limit or if the value falls even further the risk of the patient getting unconscious is high, with a possibility for fall is high resulting in the emergency mechanism generating an alert to the respective professionals.

Blood pressure monitoring is done via the PTT approach (Pulse Transit Time) using the Photo-plethysmography (PPG) sensor 118 used to monitor Blood Oxygen Saturation and Heart Rate. Its alert mechanism is bi-directional and is used only as an indicator for an alert and not as an actual alert. When the systolic and diastolic pressure values (systole & diastole) are within acceptable limits, it is considered normal. But when the values go above or below the set range, the alert mechanism is raised and the event is recorded.

The present invention therefore provides a greater accuracy of vital information without compromising the wearers' mobility, in contrast to the generally medical equipment's which take vital data from patients with the expense of user mobility. Further, in the garment form factor 24/7 vital monitoring is also achieved.

The invention achieves periodical vital reading without human assistance, thereby providing the wearer to be constantly under medical care while carrying out the everyday activities. In addition, the invention also provides the ability to be powered & function remotely to ensure cross validation of the alerts generated from the invention. The ‘round-the-clock’ data collection by the invention enables the medical professionals to understand behavioural pattern and trend analysis of live vitals. The invention helps for differential diagnosis of chronic illness and allows tracking long term medicine effect and side effects on a patient.

The invention provides an automatic emergency alert mechanism that notifies the nearest emergency team, doctor and the wearer's next of kin in event of an emergency, thus, effectively reducing the lead time taken for the emergency signal to reach the respective professionals drastically.

Even further, the garment form factor of the present invention enables everyday usability with minimal effort, thereby limiting the necessity of a medical technician for installation only. With inclusion of several bio-compatible materials for its construction, the present invention provides the capability of recycling the present invention as eco-friendly.

The present device does not compromise on the wearers' mobility to ensure round-the-clock monitoring. The device is configured to receive analog signal at the highest feasible resolution and sampling rate for continuous transmission or buffering whenever necessary. This provides the same or equivalent level of output conformity to assessment standards as the one provided by the conventional health monitoring equipment. The invention also eliminates external interferences, noise and motion artefacts by carrying over signal processing methods from an analog medium, converting it to a digital format and processing it using a single module.

The present invention may be embodied in other specific forms without departing from its scope or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

Amended claims:

1. A device 100 for measuring physiological parameters of a user, the device comprising:
a sensor unit 116 having a plurality of sensors in one or more sensor types to sense one or more physiological parameters of a user;
a control unit 102 coupled with the sensor unit 116, the control unit 102 configured to receive one or more sensed signals from each of the plurality of sensors;
a communication module 136 coupled with the control unit 102, the communication module 136 configured to establish communication between the control unit and a remote monitoring unit;
an amplifier unit 134 coupled with the control unit 102, the amplifier unit 134 configured to amplify audio signals received from at least one audio capturing instrument; and
a battery receiver and power regulation module 126 having a battery unit and a battery management system coupled with the control unit 102, and the sensor unit 116, wherein the battery receiver and power 20 regulation module 126 controls powering, charging and protection of components of the device,
one or more IMU sensors positioned in a manner to obtain one or more independent reference planes for comparison with each other to create and store an internal virtual reference plane to constitute the basis of triangulation capabilities,
wherein the control unit includes a clock generator, configured to manage timed activation and deactivation of individual measuring components, and receives physiological signals of the user through the sensor unit 116 and process the same to provide values against each of the physiological parameters to the remote monitoring unit in real-time, wherein the device 100 is adapted to be integrated in a wearable garment with all components of the device 100 embedded within said wearable garment to measure at least one of heart rhythm and electrical activity, triaxial linear and angular motion, core body temperature and blood pressure.

2. The device as claimed in claim 1, wherein the sensor unit 116 comprises PPG sensors, 118, ECG sensors, and CBT sensors.

3. The device as claimed in claim 1, wherein the control unit comprises at least one dedicated sub-control unit for each sensor type.

4. The device as claimed in claim 1, wherein the control unit comprises at least one filter controller.

5. The device as claimed in claim 1, wherein the amplifier unit comprises at least one analog to digital converter, wherein the amplifier unit is configured to receive audio signals from at least one of a full pick-up piezo-mic and at least one piezo-mic stethoscope.

6. The device as claimed in claim 5, wherein the at least one piezo-mic stethoscope is embedded into the wearable garment.

7. The device as claimed in claim 1, wherein the control unit includes at least one alert generation module configured to raise an alarm in case at least one vital parameter reading is beyond an acceptable range for a predefined time period.

8. A clothing having the device as claimed in claim 1 embedded therein, the clothing adapted to cover a torso of a user and is configured to accommodate components of the device configured to acquire, monitor and transmit one or more physiological and motion related information of the user to a remote monitoring unit.

9. The clothing as claimed in claim 8, wherein the sensors are positioned strategically in the clothing and are sealed in a waterproof manner.

10. (canceled)
